(19) United States
(12) Patent Application Publication
(10) Pub. No.: US 2010/0021887 A1
(43) Pub. Date: Jan. 28, 2010

(54) PROBE FOR TAGGING VALUABLES BASED ON DNA-METAL COMPLEX

(76) Inventors: Moshe Azoulay, Kibbutz Megiddo (IL); Mariana Pokrass, Rehovot (IL); Josef Kost, Omer (IL); Riki Goldbart, Lehavim (IL); Yaakov Pollack, Omer (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 12/067,828

(22) PCT Filed: Sep. 26, 2006
(86) PCT No.: PCT/IL06/01129

§ 371 Date: Mar. 24, 2008

Related U.S. Application Data
(60) Provisional application No. 60/720,040, filed on Sep. 26, 2005.

(51) Int. Cl. C12Q 1/68

(52) U.S. Cl. 435/6

(57) ABSTRACT

Methods are disclosed involving the formation of complex DNA-Metal and the detection of the complex, such as by employing several analytical methods, e.g., X-Ray Fluorescence, FT-IR and Raman spectroscopy.

### FIELD OF THE INVENTION

The present invention is generally related to apparatus and methods for tagging or marking materials, and particularly to employing a DNA-Metal complex as a probe for tagging or marking materials.

### BACKGROUND OF THE INVENTION

Prior art methods for protecting valuable objects from theft, resale or forging, include mostly visible and invisible dyes that develop color upon contact with a developing agent. An example is European Patent EP 0 820 498 to M. J. Smith, entitled, “Developer System for Base Reactable Petroleum Fuel Markers”.

During recent years, growing knowledge in the field of biotechnology has been implemented to construct anti-counterfeit markers that are based on nucleic acids for product authenticity verification. Published US Patent Application 20050008762 proposes a method for authenticating an object using a medium comprising of nucleic acids that is applied on the tested object. For the authentication test, the nucleic acid is extracted from the object, amplified by PCR and examined by gel electrophoresis. However, such a method is limited to just qualitative analysis and the detection method (gel electrophoresis) is less sensitive. The authenticity of the product is based on the size of the DNA alone, in contrast with the method of the invention described hereinbelow which is based on specific sequence recognition following a specific complexation reaction such as hybridization.

Another example is published US Patent Application 20050045063, which proposes using a single stranded DNA molecule that is applied on the object and identified by a fluorescence reaction as a result of a contact with an identification solution comprising nucleic acids configured as molecular beacons. The fluorescence can be measured quantitatively by relatively simple devices.

### SUMMARY OF THE INVENTION

The present invention seeks to provide a novel method for employing a DNA-Metal complex as a probe for tagging or marking materials, as is described hereinbelow. The invention has many applications, such as but not limited to, as a tagging method (marker and detector) for valuables authentication test.

Methods are disclosed involving the formation of complex DNA-Metal and the detection of the complex, such as by employing several analytical methods, e.g., X-Ray Fluorescence, FT-IR and Raman spectroscopy. The marking method provides an extended range of supports for the DNA-Metal complex, i.e. nitrocellulose paper or DNA biochip. The method may be applied for testing valuables authentication and enable to identify both hybridization and metal ion concentration by a single XRF measurement.

In accordance with an embodiment of the invention, the method uses a single stranded DNA molecule that is applied on the object and identified by a fluorescence reaction as a result of a contact with identification solution that includes nucleic acids configured as molecular beacons, wherein the fluorescence can be measured quantitatively by relatively simple devices. There is no known prior art for employing DNA-metal complex as a probe for tagging or marling materials. The present invention may use DNA-metal complex as a tagging method (marker and detector) for valuables authentication test.

The incorporation of an additional element with controlled concentration to the DNA may further be quantitatively measured to complete a practical approach for an ultimate probe. This enables almost unlimited coding capability by identifying different DNA combinations by means of hybridization, as well as precise quantitative analyses. Furthermore, the specific DNA sequence may be denatured (separated to two strands) prior to marking, and renatured just on probing. This practically means that the probing would be feasible only by adding the complementary DNA strand (available solely to the marker owner). One or both strands can be marked with same or different elements or combination of elements. The analytical approach for both the qualitative and quantitative measurements may be performed by a single method such as (XRF) or multiple methods (UV, IR, X-Ray Fluorescence, FT-IR, Raman spectroscopy or DNA bio-chip technology) for both detection and analysis. For the detection of the hybrid, a specific recognition may be employed, such as free enzyme, immobilized enzyme or complementary DNA sequence immobilized on solid support.

The marking method of the invention provides an extended range of supports for the DNA-metal complex, e.g., nitrocellulose paper or DNA biochip. The method may be applied for testing valuables authentication and enables identifying both hybridization and metal ion concentration by a single XRF measurement. Furthermore, advanced bio-micro-electronic technologies may be considered for the marker detection, without the need for an external detector. Such devices could be a modified ISFET (Ion Sensitive Field Effect Transistor) or MOCSER (Molecular Controlled Semiconductor Resistor). This approach may provide an integrated solution as a detector on chip, without any need for heavy, complicated and expensive detecting system.

Complexes of the marking substance with elements can be used in liquid, aqueous and non-aqueous organic and non-organic solutions, solids (polymers) and gels. The marking substance can be removed (separated) from the product and the specific binding done in a different host. Alternatively, after hybridization, the hybrid can be separated from the medium using a specific column.

### BRIEF DESCRIPTION OF THE DRAWINGS

In the drawings:

FIG. 1 is a simplified graphical illustration of absorption spectra versus wavelength of pure DNA with the addition of Ni(NO₃)₂ at different M/P ratios as specified in the legend, in accordance with an embodiment of the present invention.

FIG. 2 is a simplified graphical illustration of the effect of Ni(NO₃)₂ on DNA migration in gel, at different M/P ratios, where at M/P=500 an apparent transformation is observed, in accordance with an embodiment of the present invention.

FIG. 3 is a simplified graphical illustration of an AFM physical image of a DNA plasmid, in accordance with an embodiment of the present invention.

FIG. 4 is a simplified graphical illustration of an XRF spectrum with identification of the marker element (peak at T1), in accordance with an embodiment of the present invention.

FIG. 5 is a simplified schematic illustration of a complex molecule DNA-Metal, in accordance with an embodiment of the present invention.

FIG. 6A is a simplified schematic structure of an ISFET, in accordance with an embodiment of the present invention, compared with the standard MOSFET.

FIG. 6B is a simplified schematic structure of a bio-micro-device, showing quantitative measurement capability, in accordance with an embodiment of the present invention.

### DETAILED DESCRIPTION OF EMBODIMENTS

DNA has been employed recently for qualitative tagging of various substances, providing endless coding combinations. See, for example, published US Patent Application 2005004063 to M. Niggemann, M. Paeschke and A. Franz-Burgholz, entitled “Marking solution for counterfeit-resistant identification of a valuable object. In the present invention, the incorporation of an additional element with controlled concentration to the DNA (or other hybrid molecules that have the ability to bind specifically one to the other, such as DNA-RNA, antigen-antibody, enzyme-substrate) may further be quantitatively measured to complete a practical approach for an ultimate probe. For DNA as an example, it enables employing almost unlimited coding capability by identifying different DNA combinations by means of hybridization. Moreover, precise quantitative analyses are feasible due to the high resolution measurement of the elements concentration by methods that can detect these elements, such as X-Ray Fluorescence (XRF). Furthermore, the specific DNA sequence may be denatured (separated to two strands) prior to marking, and renatured just on probing. This practically means that the probing would be feasible only by adding the complementary DNA strand (available solely to the marker owner). One or both strands can be marked with same or different elements or combination of elements. The analytical approach for both the qualitative and quantitative measurements may be performed by a single method such as (XRF) or multiple methods (UV, IR, Raman spectroscopy or DNA chip technology) for both detection and analysis.

In order to make the tagging even harder to forge, the specific complexation (hybridization for DNA) may be employed by extraction, precipitation of free enzyme, immobilized enzyme, or complementary DNA sequence immobilized on solid support. Complexes of the marking substance with elements can be used in liquid, aqueous and non-aqueous organic and non-organic solutions, solids (polymers) gels. The marking substance can be removed (separated) from the product and the specific binding done in a different host, or alternatively after hybridization, the hybrid can be separated from the medium using separation processes such as specific column.

In accordance with an embodiment of the invention, preparation and use of complexes, with specific elements, of molecules having high specificity to complementary molecules such as single strand DNA, binding to its complimentary DNA or RNA strand, antigen binding to its specific antibody or enzymes binding to specific substrate.

These can be prepared in different concentrations and combinations on one or both binding entities in liquids, solids or gels. The complimentary binding molecule can be “free” or immobilized on a solid support (biochip or immobilized antibody or enzyme). The complex can also be separated using a physical and chemical step such as precipitation, binding, extraction, filtration which would be followed by detection. A possible use of such complexes can be for tagging of liquids, solids gels etc.

### Experimental Methods

The system included plasmid DNA (PEGFP) of 4.7 kbps supplied by Clonetech and metal salts (NiCl₂, Ni(NO₃)₂, CuCl₂, Cu(NO₃)₂) supplied by Sigma Aldrich. The plasmid was cut once by restriction enzyme Hind III to form a linear conformation. The DNA and metal ions were mixed at different MIP (metal to phosphate group) ratios and examined for conformational alterations of the DNA molecule as a result of the metal binding.

DNA-Metal interactions were analyzed by UV spectroscopy, gel electrophoresis, AFM imaging and RT-PCR. The bound metal concentration can be analyzed quantitatively by X-Ray Fluorescence (XRF). Alternative methods for detection of the DNA-Metal complex may be FT-IR and Raman spectroscopy (see, e.g., http://www.affymetrix.com/index.affx) or hybridization on solid support such as DNA biochip or nitrocellulose paper.

### Results

The inventors processed a plasmid DNA (PEGFP) with metal salts (NiCl₂, Ni(NO₃)₂, CuCl₂, Cu(NO₃)₂). The plasmid was cut once by restriction enzyme to form a linear conformation. The DNA and metal ions were mixed at different M/P (metal to phosphate group) ratios and examined for conformational alterations of the DNA molecule as a result of the metal binding. DNA-Metal interactions were analyzed by UV spectroscopy, gel electrophoresis, AFM imaging and RT-PCR. Hybridization was performed (de-naturation and re-naturation) and observed on the complex molecules by conventional means and the bound metal concentration analyzed quantitatively by X-Ray Fluorescence (XRF). Other methods, such as DNA biochip or nitrocellulose paper for detection of the DNA-Metal complex hybridization on solid support, and implementation of a combined device as a bio-micro-electronic detector on chip, are also within the scope of the invention.

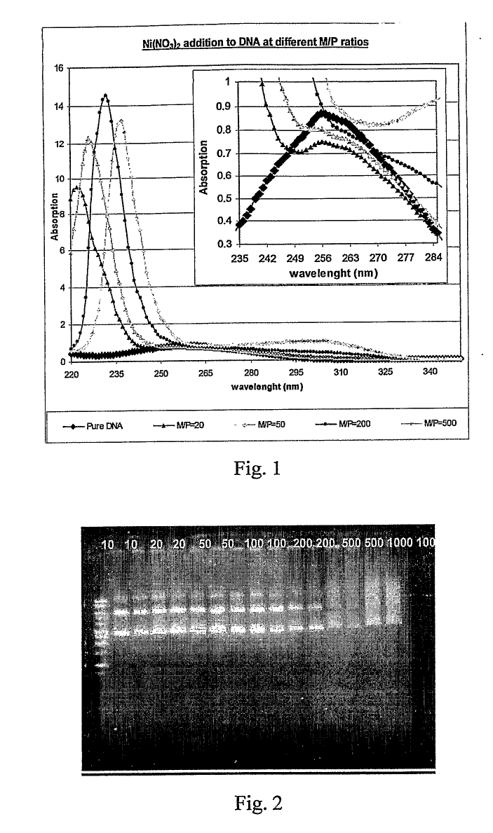

FIG. 1 illustrates the UV spectrum of a plasmid DNA and with the addition of Ni(NO₃)₂ at different M/P ratios. The DNA absorption peak at 260 nm is with good agreement to the common UV spectrum of DNA (see, e.g., Glasel, J. A. 1995. Validity of Nucleic Acid Purities Monitored by 260 nm/280 nm Absorbance Ratios. BioTechniques 18:62-63). The metal absorption spectrum ranges from 220 nm up to 250 nm depending on the concentration. Another peak of the metal is apparent at 300 nm, for which the intensity is also dependent on the concentration. It is noted that for all of the shown spectra, there are no overlaps between the DNA absorption peak at 260 nm and the metal peaks (see the magnified absorption curves on right upper side of FIG. 1). This allows the calculation of the DNA concentration by subtracting the metal spectrum.

FIG. 2 illustrates the result of gel electrophoresis for plasmid DNA with the addition of Ni(NO₃)₂ at M/P ratios ranging from 0-1000. It is noted that at M/P ratio of 500 an apparent transformation is observed. The different bands at each well are attributed to various DNA conformations (linear, relaxed and super coiled).

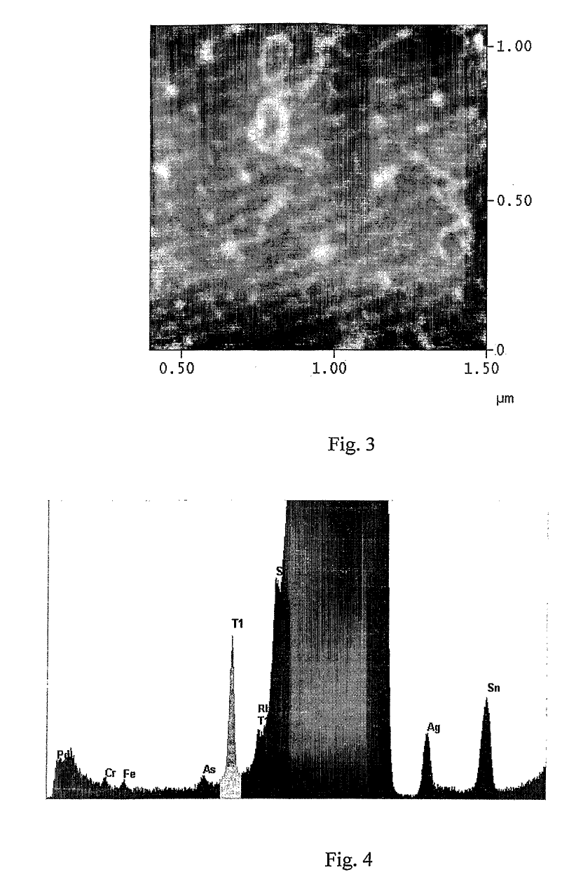

FIG. 3 illustrates a typical, high resolution, AFM (atomic force microscope) image, illustrating the physical structure of the DNA plasmid that we have employed for our study. The structure exhibited several conformations due to the incorporation of various metal concentrations.

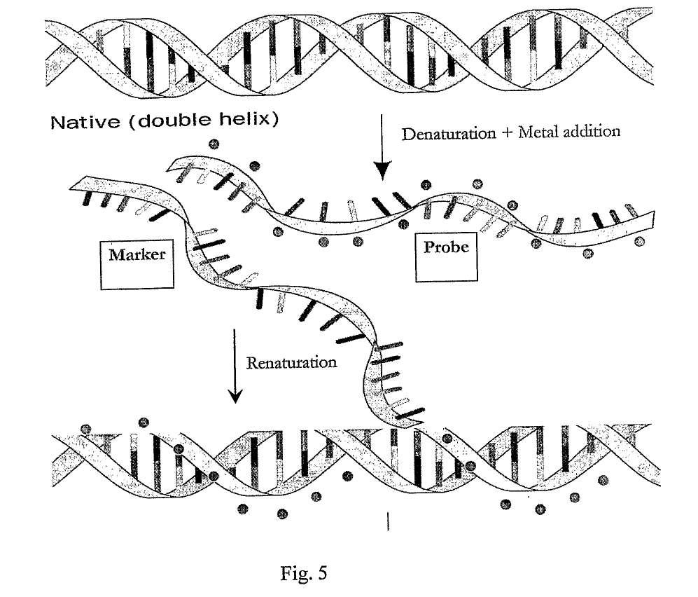

FIG. 4 illustrates a typical XRF spectrum with high precision in the identification of the marker element (green peak). The marker can be measured in a liquid solution or after binding to a solid substrate.

Reference is now made to FIG. 5. The DNA-Metal molecule may be described as two DNA helixes containing the metallic ions within the inner space of the DNA strands, as shown in FIG. 5. The marking and detection procedure involves the following steps; a) denaturizing of the DNA to two strands; b) marking one of them by reacting a single strand with metallic ions; c) tagging the substance; d) renaturizing (hybridization) the marked DNA strand with the complementary strand; e) detecting the hybridization and the metal concentration in the tagged substance.

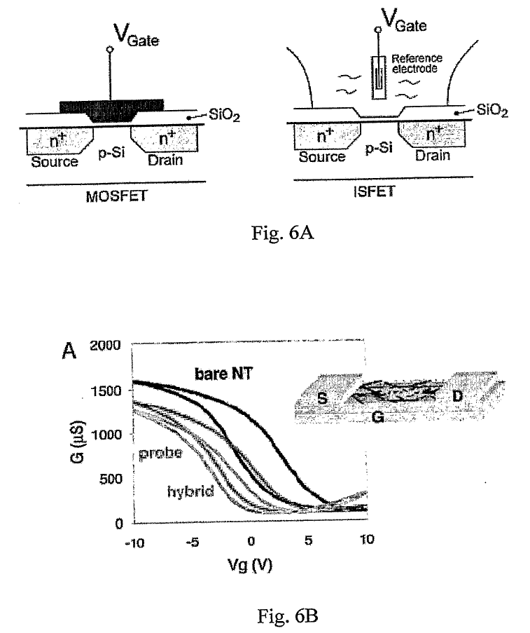

The detection method for hybridization can be performed by an integrated micro-device which is sensitive to the presence of the metallic ions in the DNA molecule that simultaneously provide a quantitative analysis of the metal concentration. A combined device is considered, consisting a bio-chip (carrying the tagged complementary DNA strand) with a modified ISFET, as seen in FIGS. 6A and 6B.

Although the invention has been described in conjunction with specific embodiments thereof, many alternatives, modifications and variations are apparent to those skilled in the art. Accordingly, all such alternatives, modifications and variations fall within the spirit and scope of the following claims.

What is claimed is:

1. A method comprising:
marking a material with a DNA-Metal complex; and
detecting said DNA-Metal complex.

2. The method according to claim 1, wherein said DNA-Metal complex is detected by an analytical method, comprising at least one of X-Ray Fluorescence, FT-IR and Raman spectroscopy, UV spectroscopy, gel electrophoresis, AFM imaging, RT-PCR and DNA bio-chip technology.

3. The method according to claim 1, further comprising using detected DNA-Metal complex to identify both hybridization and metal ion concentration in said material.

4. The method according to claim 3, wherein identifying both hybridization and metal ion concentration in said material is done by a single XRF measurement.

5. The method according to claim 1, wherein said material is marked with a single stranded DNA molecule.

6. The method according to claim 1, wherein said DNA-Metal complex is detected by a fluorescence reaction as a result of a contact with identification solution that includes nucleic acids configured as molecular beacons.

7. The method according to claim 1, further comprising denaturing a specific DNA sequence of said DNA-Metal complex prior to marking.

8. The method according to claim 7, further comprising renaturing the specific DNA sequence.

9. The method according to claim 8, wherein renaturing comprises binding a complementary DNA strand to the specific DNA sequence.

10. The method according to claim 8, wherein renaturing comprises binding a complementary RNA strand to the specific DNA sequence.

11. The method according to claim 8, wherein renaturing comprises binding an antigen to a specific antibody of the specific DNA sequence.

12. The method according to claim 8, wherein renaturing comprises binding an enzymes to the specific DNA sequence.

13. The method according to claim 1, wherein said DNA-Metal complex is disposed in a Bio-Micro-Electronic Chip.

14. The method according to claim 13, wherein said Bio-Micro-Electronic Chip comprises a modified Ion Sensitive Field Effect Transistor [Modified ISFET].
